(19) United States
(12) Patent Application Publication
(10) Pub. No.: US 2023/0240556 A1
(43) Pub. Date: Aug. 3, 2023

(54) ULTRAFAST CORONAVIRUS DETECTOR

(71) Applicants: BO GEHRING, WILLOW, NY (US); JIB RAY, PETROLIA, CA (US)

(72) Inventors: BO GEHRING, WILLOW, NY (US); JIB RAY, PETROLIA, CA (US)

(21) Appl. No.: 17/587,334

(22) Filed: Jan. 28, 2022

(51) Int. Cl. A61B 5/08; A61B 5/097; A61B 5/00

(52) U.S. Cl. CPC A61B 5/082; A61B 5/0082; A61B 5/097

(57) ABSTRACT

The COVID-19 coronavirus is detected in a subject’s breath using optical physics rather than biochemical assays.

### RELATED APPLICATION

This application claims priority to provisional patent application U.S. Serial No. 63/199,875 filed on Jan. 29, 2021 the entire contents of which are herein incorporated by reference.

### FIELD OF THE INVENTION

This invention relates to the field of medical diagnostic devices. Specifically, the present invention relates to devices which test a subject’s breath for the presence of a coronavirus such as COVID-19.

### BACKGROUND

“Currently there are two types of diagnostic tests- molecular tests, such as RT-PCR tests, that detect the virus’s genetic material, and antigen tests that detect specific proteins from the virus.” (FDA website, November 2020)

In today’s most widely used test, the antigen protein test, a swab is swirled in the subject’s nose. Nasal fluid absorbed by the swab is mixed with a reagent. The resulting mix is dripped onto a telltale strip which after a fifteen minute wait time displays a variable color to tell whether the subject is infected with COVID-19 or not.

The so-called “gold standard” RT-PCR molecular tests can take hours to display results.

The significant shortcoming of these biochemical methods is this: They cannot effectively process groups or crowds. They require minutes to hours waiting time per test. When individuals of unknown infection status wait together for their results there is high risk of contagious transmission. These biochemical methods also generate contaminated disposables such as used swabs and reagents.

In contrast, this invention is based on optical physics rather than biochemical reactions. It displays results in four seconds, more than two hundred times as fast as biochemical tests. For example, a single device can test airline passengers as quickly as they can file through a boarding gate. No waiting area is required. This invention could transform team sports, classrooms, hospital emergency rooms, religious services, workspaces, and many others where groups gather.

### DESCRIPTION OF THE INVENTION

The preferred embodiment of the invention is a battery-powered sealed device about the size of a computer mouse. It has an actuator button and a multicolor indicator LED (light-emitting diode). In operation, the said indicator LED lights up to show READY (repeating white strobe), TESTING (flashing blue), COVID NEGATIVE (green), and COVID POSITIVE (flashing red). A disposable tube is provided to reduce personal contact and the risk of transmitting the virus. If a subject tests positive, the device’s program switches the device off or otherwise disables it to prevent re-use until it is sterilized and reset. A USB port is provided for charging batteries and programming.

To reduce the risk of infecting subsequent subjects, a purging fan and filter are provided to cleanse the breath passage and create negative pressure inside the device to capture stray virus particles.

To run a test, the subject simply taps the actuator button and exhales for two seconds into the device’s breath passage or into a disposable tube. The indicator LED flashes blue as the test result is computed. In four seconds, it turns either GREEN for negative or FLASHING RED showing the subject is infected. In addition to this immediate visual indication, the results can be sent simultaneously via the device’s built-in Bluetooth to, for example, a linked smartphone app, along with other data which may be useful such as GPS geolocation for contact tracing.

### DETAILED DESCRIPTION OF CERTAIN EMBODIMENTS

When a COVID particle becomes airborne, it forms a protective envelope of a lipid surfactant similar to soap. Like soap bubbles, this protective envelope reflects light. The brightness of this reflection is proportional to the reflecting surface area arising from COVID concentration in the subject’s breath.

In operation, the device’s red LED light source 14 on PC Board 3 shines crosswise to the subject’s breath flowing through the breath passage. Sensor photodiodes 12 and 13 on PC Board 3 measure the intensity of the light reflected as described above. Analysis to determine whether the subject is negative or positive is computed by the CPU on PC Board 3. The test result is displayed by the indicator LED 5 and saved to non-volatile memory on PC Board 3. Data and results can be sent immediately via Bluetooth 8 as noted above to a linked smartphone app or cloud storage to assist in automated contact tracing.

It will be clear to those skilled in the art that embodiments such as device shape and construction, number and arrangement of sensors, light source LED details, purge fan details, direct radio link in addition to Bluetooth, and other such embodiments can be adjusted if needed for any of several applications within the scope of this invention.

### DRAWINGS AND THEIR DETAILED DESCRIPTIONS

FIG. 1 shows an exterior view. Visible are the outer enclosure 1, disposable tube 2, indicator LED 5, actuator button 6, USB cable 7, and purging fan assembly 9.

FIG. 2 removes the upper halves of the outer enclosure 1 and purging fan 9 to reveal the following features: PC board 3 which includes non-volatile memory and CPU, sensors 12 and 13, red LED 14, and also battery 4, Bluetooth antenna 8, breath filter 10, and purging fan motor 11.

FIG. 3 removes the disposable tube 2 and other components to clearly show the lower half of the breath passage.

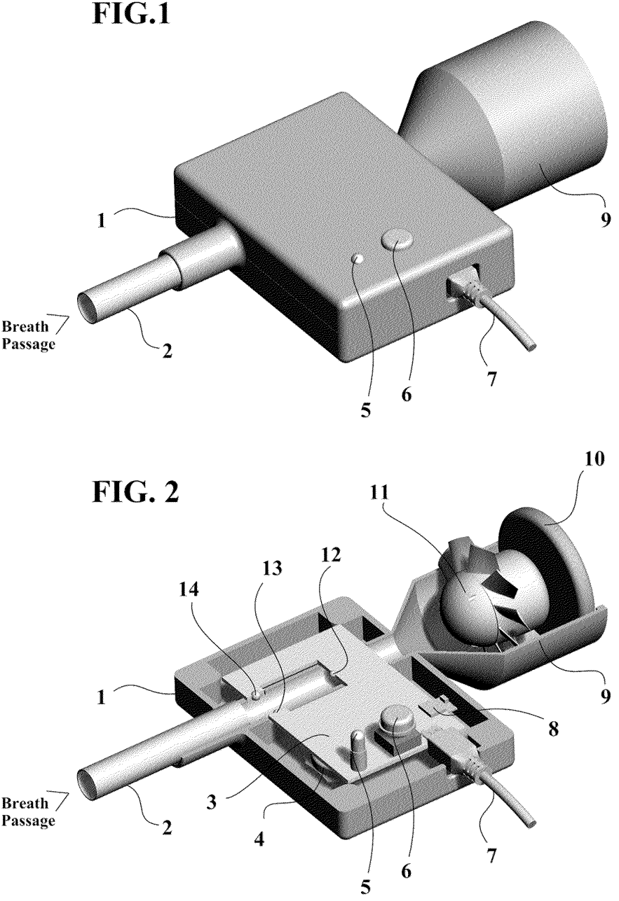

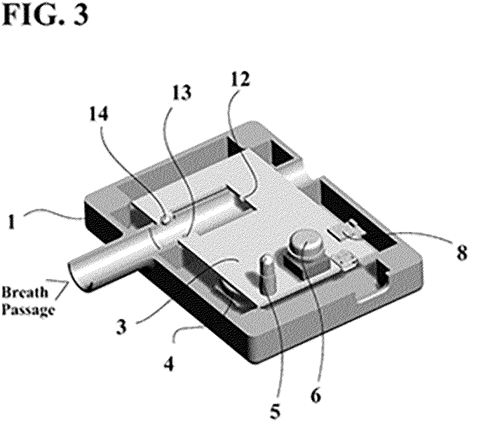

1. A device which tests a subject’s breath for the presence of a coronavirus such as COVID-19, comprising:
(a) Enclosure with a Passage through which the subject’s breath passes.
(b) Light Source positioned to shine through the said subject’s breath as the said subject’s breath passes through the said Passage.
(c) One or more Sensors positioned so as to measure the amount of light produced by said Light Source which is reflected into the Sensor(s) from coronavirus particles in the said subject’s breath.
(d) Disposable Tube inserted in the said Passage and discarded after each use.
(e) Purging Fan to cleanse said Passage of virus particles after each use.
(f) Filter to capture virus particles drawn into the said Purging Fan.
(g) Bluetooth or other wireless means to communicate externally.
